(19) United States
(12) Patent Application Publication
(10) Pub. No.: US 2007/0148658 A1
(43) Pub. Date: Jun. 28, 2007

(54) Systems and methods for biopolymeric probe design using graphical representation of a biopolymeric sequence

(76) Inventors: Charles F. Nelson, Loveland, CO (US); Amitabh Shukla, Loveland, CO (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/349,398

(22) Filed: Feb. 6, 2006

Related U.S. Application Data
(60) Provisional application No. 60/753,849, filed on Dec. 23, 2005.

(51) Int. Cl. C12Q 1/68; G06F 19/00

(52) U.S. Cl. 435/6; 702/20

(57) ABSTRACT

Systems and methods for using the same to obtairi one or more probe sequences in response to an input graphical representation of a biopolymeric sequence are provided. Also provided are computer program products for executing the subject methods.

### CROSS-REFERENCE To RELATED APPLICATION

Pursuant to 35 U.S.C. § 119 (e), this application claims priority to the filing date of U.S. Provisional Patent Application Ser. No. 60/753,849 filed on Dec. 23, 2005; the disclosure of which application is herein incorporated by reference.

### BACKGROUND OF THE INVENTION

Biopolymeric arrays (such as DNA or peptide arrays) are known and are used, for example, as diagnostic or screening tools. Such arrays include regions of usually different sequence polynucleotides or polypeptides arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon interrogating the array. For example, in an array of polynucleotide features, all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

The design and use of useful biopolymeric arrays depends upon identifying probes that meet a number of requirements, including that the probes bind to a target of interest with specificity, and fabricating the arrays in accordance with very stringent quality control criteria.

Thus, fabricating a required number of arrays, particularly with very high number of features, is often not a task an end user wishes to perform herself. As a result, array users or other customers may turn to specialized array fabricators. As the use of specialized array fabricators grows, there is continued interest in the development of improved methods performing one or more aspects of the interaction between a customer and array fabricator.

### SUMMARY OF THE INVENTION

Aspects of the subject invention include systems and methods for using the same to obtain one or more biopolymeric probes for use on an array by employing graphical visualization and selection functions. As such, embodiments of the invention provide a system for obtaining at least one biopolymeric probe sequence, where the system includes:

(a) an input manager for receiving a user biopolymer request that includes a graphical representation of a biopolymeric sequence;

(b) a probe developer for obtaining at least one probe corresponding to a selected region of the graphical representation of the biopolymeric sequence; and

(c) an output manager for providing the obtained probe to a user.

In certain embodiments, the system further includes a graphical user interface (GUI) for transferring information between the system and a user.

In certain embodiments, the system further includes a graphics developer for generating at least one graphical representation of a biopolymeric sequence in response to an identifier of the sequence.

In certain embodiments, the graphics developer is configured to generate graphical representations that can be manipulated by a user.

In certain embodiments, the graphics developer is configured to automatically update the graphical representation of the biopolymeric sequence in response to a user selecting a region of the graphical representation of the biopolymeric sequence.

In certain embodiments, the probe developer is configured to obtain a probe by using a probe design algorithm.

In certain embodiments, the probe developer is configured to obtain a probe from a database.

In certain embodiments, the output manager further provides probe content and associated annotation information relating to the obtained probe corresponding to the selected region.

In certain embodiments, the system provides for remote communication between a user and the system.

In certain embodiments, the system provides for communication between a user and the system via the Internet.

In certain embodiments, the system obtains a group of probe sequences corresponding to the selected region.

In certain embodiments, the system obtains a plurality of probe groups corresponding to the selected region.

In certain embodiments, the output manager provides a user with an option to select for purchase of one or more of the obtained probes.

In certain embodiments, in response to purchasing, the one or more probe sequences are synthesized on an array.

Aspects of the invention also include methods for obtaining at least one probe corresponding to a selected region of a biopolymeric sequence, where the method includes:

(a) submitting a biopolymer request to a system of the invention, wherein the biopolymer request includes a graphical representation of a biopolymeric sequence; and

(b) obtaining at least one probe from the system in response to the request.

In certain embodiments, the method further includes viewing at least one graphical representation of the biopolymeric sequence and selecting at least one region of the graphical representation.

In certain embodiments, the biopolymeric sequence is a nucleic acid sequence.

In certain embodiments, the nucleic acid is at least a part of a chromosome.

In certain embodiments, the biopolymeric sequence is an amino acid sequence.

In certain embodiments, the obtained probe is a nucleic acid.

In certain embodiments, the obtained probe is a polypeptide.

In certain embodiments, the region is selected by highlighting the region on the graphical representation of the nucleic acid.

In certain embodiments, the graphical representation is manipulatable.

In certain embodiments, the highlighted region is represented as a distinct graphical representation from the graphical representation of the nucleic acid.

In certain embodiments, the distinct graphical representation is a distinct color.

In certain embodiments, the obtaining step includes submitting the selected region to a nucleic acid probe database.

In certain embodiments, the obtaining step includes submitting the selected region to a nucleic acid probe design algorithm.

In certain embodiments, the submitting is via the Internet.

In certain embodiments, any of the steps of the method are reiterated.

In certain embodiments, the method further includes selecting at least one of the obtained probes and saving it in a database.

In certain embodiments, the method further includes adding probes to the database.

In certain embodiments, the method further includes deleting probes from the database.

In certain embodiments, the probes obtained from different selected regions are added to the database.

In certain embodiments, the multiple regions of the biopolymeric sequence are selected on the graphical representation.

In certain embodiments, the binding location of the at least one probe is indicated graphically on the graphical representation of the selected region.

Further aspects of the methods of the invention include obtaining at least one probe corresponding to a selected region of a biopolymeric sequence, the method including:

(a) receiving a biopolymer request that comprises a graphical representation of a biopolymeric sequence; and

(b) obtaining at least one probe in response to the request.

Aspects of the invention include computer program products that include a computer readable storage medium having a computer program stored thereon that, when loaded onto a computer, operates the computer to:

a) receive a biopolymer request that comprises a graphical representation of a biopolymeric sequence; and

b) obtain at least one probe in response to the request.

### BRIEF DESCRIPTIONS OF THE DRAWINGS

FIG. 1 illustrates a substrate carrying multiple arrays, such as may be fabricated by methods of the present invention;

FIG. 2 is an enlarged view of a portion of FIG. 1 showing multiple ideal spots or features;

FIG. 3 is an enlarged illustration of a portion of the substrate in FIG. 2;

FIG. 4 schematically illustrates a representative system of the present invention;

FIG. 5 provides a functional block diagram for a graphics-based probe obtainment method according to an embodiment of the present invention;

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

By “array layout” is meant a collection of information, e.g., in the form of a file, which represents the location of probes that have been assigned to specific features of one or more array formats, e.g., a single array format or two or more array formats of an array set.

The phrase “array format” refers to a format that defines an array by feature number, feature size, Cartesian coordinates of each feature, and distance that exists between features within a given single array.

To “array display” means to produce a probe feature on a substrate surface. As such, probe content is array displayed when features are produced on an array substrate that include the probes of the probe content.

The phrase “array request information” is use broadly to encompass any type of information/data that is employed in developing an array layout, where representative types of array request information include, but are not limited to: probe content identifiers, e.g., in the form of probe sequence, gene name, accession number, annotation, etc.; array function information, e.g., in the form of types of genes to be studied using the array, such as genes from a specific species (e.g., mouse, human), genes associated with specific tissues (e.g., liver, brain, cardiac), genes associated with specific physiological functions, (e.g., apoptosis, stress response), genes associated with disease states (e.g., cancer, cardiovascular disease), etc.; array format information, e.g., feature number, feature size, Cartesian coordinates of each feature, and distance that exists between features within a given array; etc.

A “data element” represents a property of a probe sequence, which can include the base composition of the probe sequence. Data elements can also include representations of other properties of probe sequences, such as expression levels in one or more tissues, interactions between a sequence (and/or its encoded products), and other molecules, a representation of copy number, a representation of the relationship between its activity (or lack thereof) in a cellular pathway (e.g., a signaling pathway) and a physiological response, sequence similarity to other probe sequences, a representation of its function, a representation of its modified, processed, and/or variant forms, a representation of splice variants, the locations of introns and exons, functional domains etc. A data element can be represented for example, by an alphanumeric string (e.g., representing bases), by a number, by “plus” and “minus” symbols or other symbols, by a color hue, by a word, or by another form (descriptive or nondescriptive) suitable for computation, analysis and/or processing for example, by a computer or other machine or system capable of data integration and analysis.

As used herein, the term “data structure” is intended to mean an organization of information, such as a physical or logical relationship among data elements, designed to support specific data manipulation functions, such as an algorithm. The term can include, for example, a list or other collection type of data elements that can be added, subtracted, combined or otherwise manipulated.

Exemplarily, types of data structures include a list, linked-list, doubly linked-list, indexed list, table, matrix, queue, stack, heap, dictionary, flat file databases, relational databases, local databases, distributed databases, thin client databases and tree. The term also can include organizational structures of information that relate or correlate, for example, data elements from a plurality of data structures or other forms of data management structures. A specific example of information organized by a data structure of the invention is the association of a plurality of data elements relating to a gene, e.g., its sequence, expression level in one or more tissues, copy number, activity states (e.g., active or non-active in one or more tissues), its modified, processed and/or and/or variant forms, splice variants encoded by the gene, the locations of introns and exons, functional domains, interactions with other molecules, function, sequence similarity to other probe sequences, etc. A data structure can be a recorded form of information (such as a list) or can contain additional information (e.g., annotations) regarding the information contained therein. A data structure can include pointers or links to resources external to the data structure (e.g., such as external databases). In one aspect, a data structure is embodied in a tangible form, e.g. is stored or represented in a tangible medium (such as a computer readable medium).

The term “object” refers to a unique concrete instance of an abstract data type, a class (that is, a conceptual structure including both data and the methods to access it) whose identity is separate from that of other objects, although it can “communicate” with them via messages. In some occasions, some objects can be conceived of as a subprogram which can communicate with others by receiving or giving instructions based on its, or the others' data or methods. Data can consist of numbers, literal strings, variables, references, etc. In addition to data, an object can include methods for manipulating data. In certain instances, an object may be viewed as a region of storage. In the present invention, an object typically includes a plurality of data elements and methods for manipulating such data elements.

A “relation” or “relationship” is an interaction between multiple data elements and/or data structures and/or objects. A list of properties may be attached to a relation. Such properties may include name, type, location, etc. A relation may be expressed as a link in a network diagram. Each data element may play a specific “role” in a relation.

As used herein, an “annotation” is a comment, explanation, note, link, or metadata about a data element, data structure or object, or a collection thereof. Annotations may include pointers to external objects or external data. An annotation may optionally include information about an author who created or modified the annotation, as well as information about when that creation or modification occurred. In one embodiment, a memory comprising a plurality of data structures organized by annotation category provides a database through which information from multiple databases, public or private, may be accessed, assembled, and processed. Annotation tools include, but are not limited to, software such as BioFerret (available from Agilent Technologies, Inc., Palo Alto, Calif.), which is described in detail in application Ser. No. 10/033,823 filed Dec. 19, 2001 and titled “Domain-Specific Knowledge-Based Metasearch System and Methods of Using.” Such tools may be used to generate a list of associations between genes from scientific literature and patent publications.

As used herein an “annotation category” is a human readable string to annotate the logical type the object comprising its plurality of data elements represents. Data structures that contain the same types and instances of data elements may be assigned identical annotations, while data structures that contain different types and instances of data elements may be assigned different annotations.

As used herein, a “probe sequence identifier” or an “identifier corresponding to a probe sequence” refers to a string of one or more characters (e.g., alphanumeric characters), symbols, images or other graphical representation(s) associated with a probe sequence comprising a probe sequence such that the identifier provides a “shorthand” designation for the sequence. In one aspect, an identifier comprises an accession number or a clone number. An identifier may comprise descriptive information. For example, an identifier may include a reference citation or a portion thereof.

As used herein “probe request information” refers to any type of information that is employed to obtain one or more probes, and may comprise one or more search terms, key words, accession numbers, or probe sequences. Probe request information may take a number of different forms, such as sequence information, location identifier information, art accepted identifier, e.g., accession no, information, etc. Likewise, probe content information may take a number of different forms, such as sequence information, location identifier information, art accepted identifier, e.g., accession no, information, etc. In one aspect, “probe content information” includes a probe sequence or an identifier associated therewith, structural, and functional genomic and/or proteomic information with respect to the probe sequence and/or identifier. In another aspect, probe content information is relevant links to reagents or kits that might be used to obtain additional probe content information (e.g., such as links to sources of primers, antibodies, binding partners, and host cells, including transgenic animals expressing the sequences or modified forms there of, and the like). In other aspects, probe content information may include, but is not limited to information regarding cell(s) or tissue(s) in which a probe sequence is expressed and/or levels of expression, information concerning physiological responses of a cell or tissue in which the sequence is expressed (e.g., whether the cell or tissue is from a patient with a disease), chromosomal location information, copy number information, information relating to similar sequences (e.g., homologous, paralogous or orthologqus sequences). Additional probe content information can include frequency of the sequence in a population, information relating to polymorphic variants of the probe sequence (e.g., such as SNPs), information relating to splice variants (e.g., tissues, individuals in which such variants are expressed), and or demographic information relating to individual(s) in which the sequence is found.

The phrase “best-fit” refers to a resource allocation scheme that determines the best result in response to input data. The definition of ‘best’ may vary depending on a given set of predetermined parameters, such as sequence identity limits, signal intensity limits, cross-hybridization limits, Tm, base composition limits, probe length limits, distribution of bases along the length of the probe, distribution of nucleation points along the length of the probe (e.g., regions of the probe likely to participate in hybridization, secondary structure parameters, etc. In one aspect, the system considers predefined thresholds. In another aspect, the system rank-orders fit. In a further aspect, the user defines his or her own thresholds, which may or may not include system-defined threshold. \A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that many computer-based systems are available which are suitable for use in the present invention. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, UBS, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

Items of data are “linked” to one another in a memory when the same data input (for example, filename or directory name or search term) retrieves the linked items (in a same file or not) or an input of one or more of the linked items retrieves one or more of the others.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g;, a nucleotide base, etc.). In the art synthesis of nucleic acids of this type utilizes an initial substrate-bound monomer that is generally used as a building-block in a multi-step synthesis procedure to form a complete nucleic acid. A “biomonomer” references a single unit, which can be linked with the same or other biomonomers to form a biopolymer (e.g., a single amino acid or nucleotide with two linking groups, one or both of which may have removable protecting groups).

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise about 2-50 monomers, preferably about 2-20, more preferably about 3-10 monomers.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

A “biopolymer” is a polymer of one or more types of repeating units. Biopolymers are typically found in biological systems (although they may be made synthetically) and may include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” may include DNA (including cDNA), RNA, oligonucleotides, and PNA and other polynucleotides as described in U.S. Pat. No. 5,948,902 and references cited therein (all of which are incorporated herein by reference), regardless of the source.

The term “biomolecule” means any organic or biochemical molecule, group or species of interest that may be formed in an array on a substrate surface. Exemplary biomolecules include peptides, proteins, amino acids and nucleic acids.

The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample.

The term “ligand” in the context of the invention may or may not be an “oligomer” as defined above. However, the term “ligand” as used herein may also refer to a compound that is “pre-synthesized” or obtained commercially, and then attached to the substrate.

The term “sample” as used herein relates to a material or mixture of materials, typically, although not necessarily, in fluid form, containing one or more components of interest.

A biomonomer fluid or biopolymer fluid reference a liquid containing either a biomonomer or biopolymer, respectively (typically in solution).

The term “peptide” as used herein refers to any polymer compound produced by amide formation between an α-carboxyl group of one amino acid and an a-amino group of another group.

The term “oligopeptide” as used herein refers to peptides with fewer than about 10 to 20 residues, i.e., amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 up to about 200 nucleotides in length, e.g., from about 25 to about 200 nt, including from about 50 to about 175 nt, e.g. 150 nt in length

The term “polynucleotide” as used herein refers to single- or double-stranded polymers composed of nucleotide monomers of generally greater than about 100 nucleotides in length.

An “array,” or “chemical array” used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. As such, an addressable array includes any one or two or even three-dimensional arrangement of discrete regions (or “features”) bearing particular biopolymer moieties (for example, different polynucleotide sequences) associated with that region and positioned at particular predetermined locations on the substrate (each such location being an “address”). These regions may or may not be separated by intervening spaces. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either precursor units (such as nucleotide or amino acid monomers) in the case of in situ fabrication, or the previously obtained biomolecule, e.g., polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323;043, U.S. patent application Ser. No. 09/302,898 filed Apr. 30, 1999 by Caren et al., and the references cited therein. Other drop deposition methods can be used for fabrication, as previously described herein.

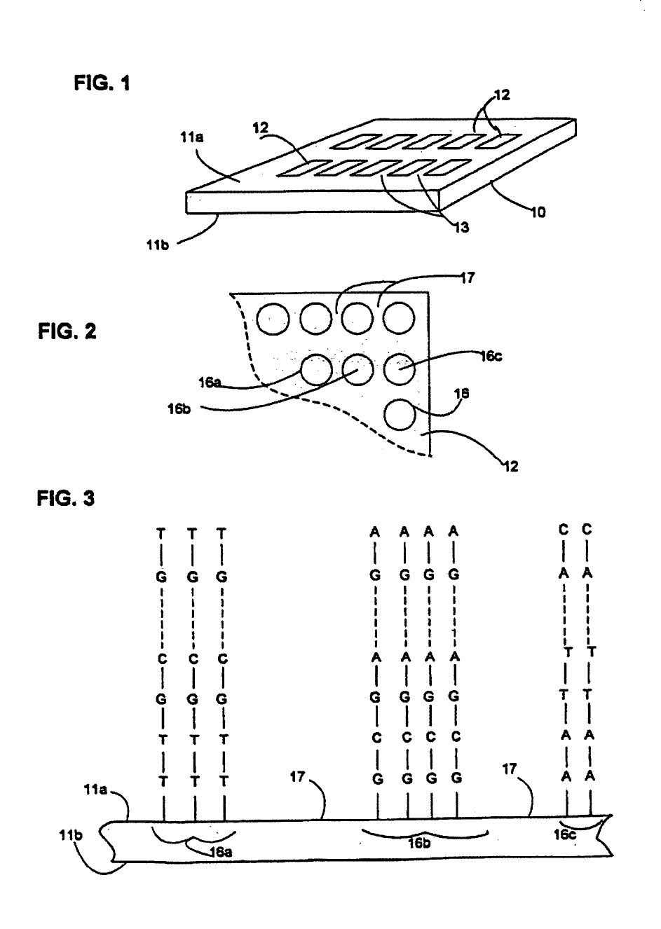

An exemplary chemical array is shown in FIGS. 1-3, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the surface 111b, with regions of the rear surface 111b adjacent the opposed sides 113c, 113d and leading end 113a and trailing end 113b of slide 110, not being covered by any array 112. A second surface 111a of the slide 110 does not carry any arrays 112. Each array 112 can be designed for testing against any type of sample; whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymer ligands, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

The substrate may be porous or non-porous. The substrate may have a planar or non-planar surface.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “web” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1'SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions.include hybridization at 60° C, or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“Contacting” means to bring or put together. As such, a first item is contacted with a second item when the two items are brought or put together, e.g., by touching them to each other.

“Depositing” means to position, place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

“Biopolymer request comprising a graphical representation of a biopolymeric sequence” means an input by a user of a system of the invention in the form of a graphical representation of a biopolymer for the purpose of obtaining at least one probe that corresponds to the biopolymer. In certain embodiments,.the biopolymer request is a selected region of a graphical representation of a biopolymer.

### DETAILED DESCRIPTION OF THE INVENTION

As summarized above, systems and methods for using the same to obtain one or more probe sequences in response to an input graphical representation of a biopolymeric sequence are provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, aspects of the invention include systems and methods of using the same which may be employed to obtain probes that correspond to a selected region of a graphical representation of a biopolymeric sequence. In further describing the aspects of the invention, a review of representative system hardware/software architecture is provided, followed by a more detailed discussion of aspects-of representative embodiments of the invention.

### Systems

Representative embodiments of the subject systems generally include the following components: (a) an input manager for receiving a user biopolymer request comprising a graphical representation of a biopolymeric sequence; (b) a probe developer for obtaining at least one probe corresponding to a selected region of the graphical presentation of the biopolymeric sequence; and (c) an output manager for providing the at least one obtained probe. In certain embodiments, the system includes a graphical user interface (GUI) to facilitate communication between the system and a user. In certain embodiments, the system may include other functional components as described in more detail below (e.g., graphics developer, session manager, etc.).

The system includes a memory component, which may be any of a variety of known or future memory storage devices. Examples include any commonly available random access memory (RAM), magnetic medium such as a resident hard disk or tape, an optical medium such as a read and write compact disc, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In certain embodiments, a computer program product is described comprising a computer usable medium having control logic (computer software program, including program code) stored therein. The control logic, when executed by the processor the computer, causes the processor to perform functions described herein. In certain other embodiments, some functions are implemented primarily in hardware using, for example, a hardware state machine. Implementation of the hardware state machine so as to perform the functions described herein will be apparent to those skilled in the relevant arts.

The input-output controllers of the computer could include any of a variety of known devices for accepting and processing information from a user whether a human or a machine, whether local or remote. Such devices may include, for example, modem cards, network interface cards, sound cards, or other types of controllers for any of a variety of known input devices. Output controllers of input-output controllers could include controllers for any of a variety of known display devices for presenting information to a user, whether a human or a machine, whether local or remote. If one of the display devices provides visual information, this information typically may be logically and/or physically organized as an array of picture elements, sometimes referred to as pixels. A graphical user interface (GUI) controller may comprise any of a variety of known or future software programs for providing graphical input and output interfaces between the computer (510) and a user, and for processing user inputs. In one aspect, the system may include a plurality of graphical user interfaces for viewing and manipulating multiple sets of data. In another aspect, the system will automatically provide modified information (e.g., such as graphical representations of biopolymeric sequences) to other permitted users of the system. The functional elements of the computer 510 may communicate with each other via system bus. Some of these communications may be accomplished in alternative embodiments using network or other types of remote communications.

In representative embodiments, the subject systems may be viewed as being the physical embodiment of a web portal, where the term “web portal” refers to a web site or service, e.g., as may be viewed in the form of a web page, that offers a broad array of resources and services to users via an electronic communication element, e.g., via the Internet. Each of these elements is described in greater detail below.

The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen: Appropriate operating systems include Windows NT®, Sun Solaris, Linux, OS/400, Compaq Tru64 Unix, SGI IRIX, Siemens Reliant Unix, and others.

In certain embodiments, the subject systems include multiple computer platforms which may provide for certain benefits, e.g., lower costs of deployment, database switching, or changes to enterprise applications, and/or more effective firewalls. Other configurations, however, are possible. For example, as is well known to those of ordinary skill in the relevant art, so-called two-tier or N-tier architectures are possible rather than the three-tier server-side component architecture represented by, for example, E. Roman, Mastering Enterprise JavaBeans™ and the Java™2 Platform (John Wiley & Sons, Inc., NY, 1999) and J. Schneider and R. Arora, Using Enterprise Java. (Que Corporation, Indianapolis, 1997).

It will be understood that many hardware and associated software or firmware components that may be implemented in a server-side architecture for Internet commerce are known and need not be reviewed in detail here. Components to implement one or more firewalls to protect data and applications, uninterruptable power supplies, LAN switches, web-server routing software, and many other components are not shown. Similarly, a variety of computer components customarily included in server-class computing platforms, as well as other types of computers, will be understood to be included but are not shown. These components include, for example, processors, memory units, input/output devices, buses, and other components noted above with respect to a user computer. Those of ordinary skill in the art will readily appreciate how these and other conventional components may be implemented.

The functional elements of system may also be implemented in accordance with a variety of software facilitators and platforms (although it is not precluded that some or all of the functions of system may also be implemented in hardware or firmware). Among the various commercial products available for implementing e-commerce web portals is BEA WebLogic from BEA Systems, which is a so-called “middleware” application. This and other middleware applications are sometimes referred to as “application servers,” but are not to be confused with application server hardware elements. The function of these middleware applications generally is to assist other software components (such as software for performing various functional elements) to share resources and coordinate activities. The goals include making it easier to write, maintain, and change the software components; to avoid data bottlenecks; and prevent or recover from system failures. Thus, these middleware applications may provide load-balancing, fail-over, and fault tolerance, all of which features will be appreciated by those of ordinary skill in the relevant art.

Other development products, such as the Java™2 platform from Sun Microsystems, Inc. may be employed in the system to provide suites of applications programming interfaces (API's) that, among other things, enhance the implementation of scalable and secure components. Various other software development approaches or architectures may be used to implement the functional elements of system and their interconnection, as will be appreciated by those of ordinary skill in the art.

In certain embodiments, the system comprises a communication module for communicating with a graphical user interface (e.g., to create first instances of objects in a memory of the system and to output displays that allow a user to interact with the system and obtain information about data elements associated with an object). The system may be deployable using any operating system known in the art, such as Windows XP. In certain aspects, the system executes one or more programs that run on a Web server and build Web pages. In another aspect, the system is capable of building a Web page on the fly allowing the system to dynamically adapt to a user's requests. In still a further aspect, static HTML may be mixed with dynamically-generated HTML for this purpose. The system may include typical browsers known in the art such as Internet Explorer 4.0+ and Netscape Navigator 6.0+.

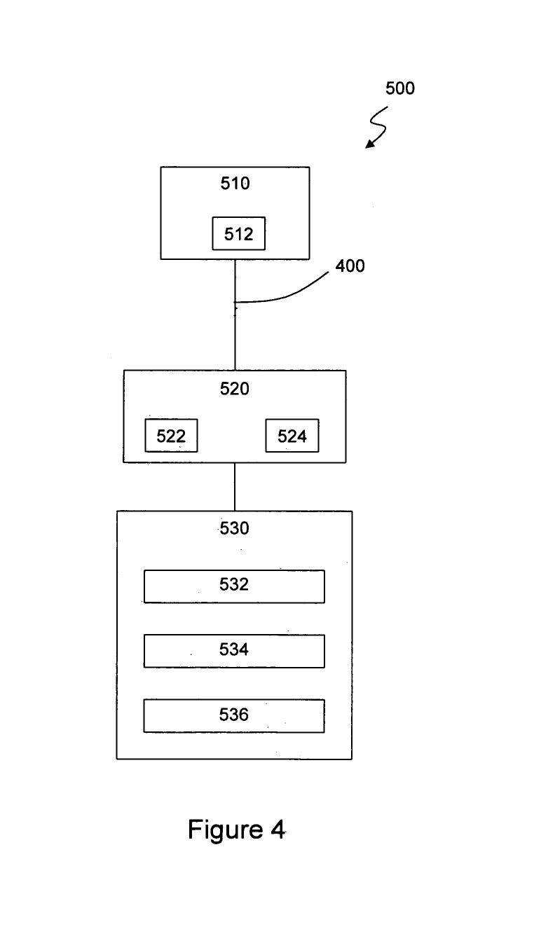

FIG. 4 provides a view of a representative system according to an embodiment of the subject invention. In FIG. 4, system 500 includes a computer 510 with graphical user interface (GUI) 512, communications module 520 and processing module 530. Each module may be present on the same or different platforms, e.g., servers, as described above.

As shown in FIG. 4, computer 510 is coupled via network cable 400 to the communications module 520. Additional computers of other users in a local or wide-area network including an Intranet, the Internet, or any other network may also be coupled to communications module 520 via cable 400. It will be understood that cable 400 is merely representative of any type of network connectivity, which may involve cables, transmitters, relay stations, network servers, and many other components not shown but evident to those of ordinary skill in the relevant art. Via user computer 510, a user may operate a web browser served by a user-side. Internet client to communicate via Internet with system 500. System 500 may similarly be in communication over Internet with other users and/or networks of users, as desired.

A representative embodiment of a system of the invention is shown in FIG. 4. It is to be understood that this representative system is not limiting, as other embodiments of the invention may including fewer or additional components as desired.

Communications module (520) is operatively connected to computer (510) providing a vehicle for a user to interact with the components of the system (500). The computer may be specially designed and configured to support and execute any of a multitude of different applications. The computer also may be any of a variety of types of general-purpose computers such as a personal computer, network server, workstation, or other computer platform now or later developed. In addition to a GUI (512), the computer in these embodiments typically includes known components such as a processor, an operating system, a system memory, memory storage devices, and input-output controllers. It will be understood by those skilled in the relevant art that there are many possible configurations of the components of a computer in these embodiments and that some components are not listed above, such as cache memory, a data backup unit, and many other devices. The processor maybe a commercially available processor such as a Pentium® processor made by Intel Corporation, a SPARC® processor made by Sun Microsystems, or it may be one of other processors that are or will become available. The processor executes the operating system, which may be, for example, a Windows®-type operating system (such as Windows NT®4.0 with SP6a) from the Microsoft Corporation; a Unix® or Linux-type operating system available from many vendors; another or a future operating system; or some combination thereof. The operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

Communications module (520) includes input manager (522) which is configured to receive, process and forward information from computer 510, e.g., over the Internet. For example, input manager (522) processes and forwards biopolymeric sequence information input from a user through computer (510) to the processing module 530 for further processing, as described below. These functions are performed in accordance with known techniques common to the operation of Internet servers, also commonly referred to in similar contexts as presentation servers.

Another of the functional elements of communications module (520) is output manager (524). Output manager 524 provides information assembled by processing module (530), described below, to a user, e.g., over the Internet, also in accordance with those known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources.

The system of the present invention includes a processing module (530) comprising various functional elements that carry out specific tasks on the platforms in response to information submitted into the system by one or more users. In FIG. 4, elements 532, 534 and 536 represent three different functional elements of processing module (530). While three different functional elements are shown, it is noted that the number of functional elements may be more or less depending on the particular embodiment of the invention. Elements of the processing module are now reviewed in greater detail.

In certain embodiments, the processing module (530) of the invention includes a graphics developer (532) which generates graphical representations of biopolymeric sequences of interest to the user. The graphic representations generated by graphics generator (532) can be any of a variety of types and can represent primary, secondary, and/or tertiary structural features of a biopolymeric sequence. By primary structural feature is meant the sequential organization of monomers that make up the biopolymer; by secondary structural feature is meant the interaction of the monomers of the primary sequence as represented in a two dimensional context (e.g., the representation of a hairpin loop in a nucleic acid sequence as a “lasso-like” structure), and by tertiary structural feature is meant the three dimensional structure of a biopolymeric sequence (e.g., beta sheets of a protein and/or other structural features).

In certain embodiments, the graphics developer (532) generates more than one graphical representation for a given biopolymeric sequence. For example, if a chromosome of interest is identified by a user (meaning that the user provides an indication to the system that probes are sought for a region of a specific chromosome), the graphics developer may generate one graphical representation that corresponds to the entire sequence of the chromosome and another that represents a sub-section of the sequence (e.g., a region containing a specific gene).

In certain embodiments, the graphical representation generated by the graphics developer is able to be updated either by instruction from the user (e.g., is manipulable) or automatically by the graphics developer using pre-programmed instructions. Updating the graphical representation can include any number of alterations to the graphical representation including, but not limited to, changing the color, the orientation, the portion displayed (e.g., displaying only the user-selected region), changing between primary, secondary, or tertiary structural views, updating annotations of the graphical representation (e.g., indicating the genes present on the current view of a graphical representation of a chromosome), etc. In certain embodiments, the graphical representation can be manipulated by the user using any number of standard graphics manipulation functions known in the computer graphics art, including (but not limited to) zooming, rotating, panning, and flipping functions.

The graphical representations generated by graphics developer (532) are selectable, meaning that a user is able to select all or a part of the graphical representation using GUI (512) for which corresponding biopolymeric probes are sought. In certain embodiments, selection by the user of all or a portion of a graphical representation of a biopolymeric sequence results in a distinct change in appearance of the selected region so that the user can easily distinguish the selected region from non-selected region. In certain embodiments, the distinct change in appearance of the selected region is a change of color. In certain other embodiments, an additional graphical view representing only the selected region is generated by the graphics developer and displayed together with the graphical representation of the entire parental biopolymeric sequence. As discussed in more detail below, the graphics developer (532) can be configured in many different ways depending on the specific requirements of the user of the system.

The processing module (530) of the invention includes a biopolymeric sequence extractor (534). This element is configured to extract the biopolymeric sequence from the user-selected region of the graphical representation for use in obtaining probes for that region.

In certain embodiments, processing module (530) also includes a probe developer (536) configured to obtain a probe sequence (or a plurality of probe sequences) that correspond to a region of interest selected by a user on the graphical representation generated by graphics developer (532). In a representative embodiment, probe developer (536) receives a biopolymeric sequence (or sequences) from biopolymeric sequence extractor (534). As described above, the biopolymeric sequence extractor (534) extracts the biopolymeric sequence of a region for which probes are sought, the region being selected by a user on a graphical representation of a parent biopolymeric sequence. Once receiving a sequence, probe developer (536) obtains a suitable probe (or probes) for the user-selected region. The probe developer may obtain probe sequences in any of a variety of ways, including by designing them using probe design programs, by retrieving pre-designed and/or validated probe sequences from a database that correspond to the selected region, or a combination of the two.

In certain embodiments, the probe developer of the processing module is configured to provide at least one probe sequence in response to a graphical selection submitted by a user. The probe developer may be configured to provide sequences for probes directed to regions of the selected region that are spaced at predetermined finite distances from each other. For example, in embodiments in which the biopolymer is a nucleic acid, the returned probes may be spaced every 50 bases, 100 bases, every 1000 bases, every 5,000 bases etc., over the selected region.

In certain embodiments, the probe developer of the processing module is configured to provide at least one second probe sequence in response to request information that comprises identifier information for a first probe sequence, wherein the second probe sequence shares sequence identity with the first probe. In certain of these embodiments, the amount of sequence identity is at least about 25%, at least about 30%, at least about 40%, at least about 50%, at least about 60%, at least about 70%, at least about 80%, at least about 90% or higher, as desired. In certain of these embodiments, the probe developer of the processing module is configured to provide at least one homologous probe sequence from a second species in response to request information that comprises identifier information for a probe from a first species. As above, the identifier information may be a particular sequence of a probe used to assay target from a first species, or a non-sequence based identifier, e.g., an art accepted name of a gene of interest, an accession no., etc. The probe developer receives the probe information for the first sequence and, based therein, returns one or more probe sequences for a second species of interest. In developing such probes, the probe developer may search stored probe sequences from species different from the first species, and/or search other databases of probe sequences, which may be either public or private. The returned homologous probes may be orthologs or paralogs, depending on their classification by the art. For example, a user may enter one or more mouse probe sequences into the system and request corresponding human probe sequences for the mouse probe sequences, where the human probe sequences are sequences that are homologous to the mouse probe sequences, e.g., are orthologs or paralogs of the mouse sequences. In certain embodiments, the user may enter multiple probe sequences for use with a given first species, e.g., mouse, and request corresponding multiple probe sequences for another species, e.g., human. The probe developer provides the requested multiple probe sequences from the second species in response to this request. In certain embodiments, the request for multiple probe sequences is made by submitting an array layout for the first species, and requesting a corresponding array layout for the second species. This representative embodiment is illustrated in FIG. 11.

In certain embodiments, the probe developer of the processing module is configured to provide a probe set of high-resolution probe sequences in response to request information that comprises identifier information for a low-resolution probe.

In one aspect, the term “high-resolution probe” refers to one or more probes that elucidate small differences between populations and/or treatment groups in microarray experiments in comparison to a “low-resolution probe” to that elucidates large differences between populations and/or treatment groups in microarray experiments. In another aspect, a “high-resolution probe” refers to a probe that can be used to scan smaller regions of a genome relative to a low-resolution probe, which can be used to scan larger regions of a genome.

In these embodiments, a user inputs into the system an identifier for a first probe, as described above, where the identifier is typically a sequence of a given target nucleic acid or some other identifier that identifies a particular location of a target nucleicacid, e.g., an mRNA or a genomic region of DNA. The probe developer then identifies a plurality of probes having a predetermined distance on the target nucleic acid from the location of the target nucleic acid specified by the identifier. For example, the probe developer may generate a series of X probes of N bases in length that are positioned on the target nucleic acid at increasing intervals, spaced uniformly or non-uniformly as desired (such as every 1000 bases) from the input location. This series of output probes may be viewed as a series of high-resolution probes with respect to the input, low resolution probe. An example of where this feature is of interest is in array-based CGH applications. In such applications, a user may perform a first assay using a low-resolution array i.e., an array having features that span an entire genome of a species, such that the probes hybridized to regions of the species chromosome that are separated from each other by large distances. Probes of interest that are identified in this first, low-resolution assay, may then be input by a user into the probe developer. The probe developer will then return to the user a series of high-resolution probes relative to the input probe, where the user may use these probes in a probe layout for use in a second, higher resolution array-based CGH assay. The probe developer may allow the user to specify certain parameters for developing the higher resolution set, such as number of probes to be produced, interval distance between probes, and the like.

The probe developer may also be used to generate probes that scan user-selected regions of the genome (which may be high or low resolution probes). For example, probe information request supplied by the user can include, but is not limited to, desired distances of probe targets from a reference point (e.g., the centromere, a chromosomal band, a gene, a chromosomal abnormality, and the like), selection of all available probes in a user-defined region, number of probes per given sequence, requests for probes that include desired regulatory regions, protein-binding sites, RNA-binding sites, methylation sites, splice regions, combinations thereof and the like. In one aspect, the system provides menu options, check boxes, and the like, providing categories of types of probe information request and allowing a user to select desired types. Alternatively, or additionally, the system may allow a user to input values into one or more fields associated with categories of probe request information, allowing the user to define with more particularity, desired probes.

In certain embodiments, a user is able to select probes that work functionally well with all gene products of an identified region of interest vs. selecting probes that are distinct for a single gene product of the set of gene products of an identified region of interest. In such embodiments, a user would have the option to: (a) retrieve a probe that will work across all gene products for a selected region; (b) retrieve a probe that is distinct for the genes of a given region; or (c) select all probes returned for the region.

In certain embodiments, the probe developer of the processing module is configured to provide validation information for a probe provided in response to received array probe request information from a user. In these embodiments, a user will input probe request information into the system, as described above. The probe developer may then return to the user one or more validated probes, where a probe is considered to be validated if it has been empirically tested and shown to function according to a predetermined set of functional criteria, e.g., the probe provides a suitable signal and suitable low background noise. In addition to returning to the user one or more validated probes, the probe developer may also make available to the user, e.g., for downloading, validation information for the probe, e.g., information regarding how the probe was validated, the results the probe gave in the validation assays to which it was subjected, and the like. Such validation information may be employed by the user in a number of different manners, e.g., to support results obtained using an array that includes the corresponding validated probe.

In certain embodiments, the probe developer of the processing module is configured to provide a probe set of normalization probes in response the array probe request information. The term “normalization probe” refers to probes that have been empirically proven to show constant signal intensities, and can be used to normalize microarray data results. In these embodiments, a user may input probe request information, in response to which the probe developer may, in addition to providing a probe based on the request information, suggest to the user one or more normalization probes to use with the provided probe. In addition, the User can define the target intensity values of the Normalization probes, and/or define a profile of a normalization probe set, where the profiles contain target intensity values that the normalization probes should exhibit. In addition, the User can define a range of specificity for the probe intensity values.

The above specifically described functions that may be performed by the probe developer are merely representative of different functions that may be performed by the probe developer. In certain embodiments, the probe developer performs two or more of the above specific functions, including three or more, four or more, five or more, as well as all of the above specific functions.

In certain embodiments, the probe developer may be configured to obtain a probe or group of probes that may not be entirely contained within the user selected region. For example, the probe developer may obtain a probe in which a portion of its length is in the region of interest and a portion is outside the region of interest. In addition, the system may return a group of probes that contains both probes that correspond to the selected region and those that do not.

In certain embodiments, in addition to the user-selected region, certain types of search criteria may be input by the user to further guide the system in obtaining probes for the selected region. These search criteria include specific biochemical attributes of the probe (e.g., approximate or precise length, affinity and/or melting temperature, specificity, etc.), the number or probes to be returned, whether the probes have been validated for use, whether the probes are commercially available, groups in which probes specific for the selected region are found, etc.

Relationships may also be used as search criteria. Basic search criteria can depend upon an object's attributes and advanced search criteria can depend upon association of the object with other objects, e.g., by searching properties of related objects. For example, attributes associated with a probe object may include unique identifier, gene function, etc. The sequence may also be represented by a Sequence object, which may include such attributes as function. So, the basic criteria for searching for a probe using the system oif the inventon is by sequence, while more advanced search criteria may also include searching for a probe by interactions with other genes/gene products in a pathway.

In certain embodiments, the system may include one or more probe object managers. In one aspect, a probe manager is a factory class for creating/copying/updating/finding/deleting probe objects. Probe managers may be created by the system on the fly e.g., for each new session in which a user interacts with the system. The system may be used to organize probe objects into probe group objects. In certain embodiments, a probe group object encapsulates a list of probes grouped together based on some criteria. A probe group object has a many-to-many relationship with a probe object. A probe group object can belong to one domain and can be shared to other domains. A probe group can have zero or more annotations associated with it. A probe group having a zero annotation, for example, may include probe groups with unknown targets.

Like probe objects, probe group objects can be associated with attributes, including, but not limited to: probe group ID, probe group name, annotations associated with the probes in the probe group, annotation category to which the annotations belong, search criteria (e.g., the search criteria used by a user to generate the probe group), status (e.g., “locked” or “in progress”), the domain to which the probe group belongs, domain share (a set of domains to which the probe group object has been shared to), number of probes in the probe group, the ID of the user who created the probe group, the date on which the probe group was created, date on which the probe group was modified (arid ID of the user who modified), and the like. Certain attributes may change over time (e.g., over sessions, as discussed further below). For example, “search criteria” is an example of a dynamic attribute that may change over time.

In response to a query, an output may be displayed by the system. For example, the output may display the obtained probes in text form (e.g., as a list or a table), in graphic form (e.g., superimposed graphically on the graphical representation of the biopolymeric sequence of interest), or both. In one aspect, the result to be shown is displayed on a Web page which includes capabilities for allowing possible actions. Such capabilities can include, but are not limited to, links, buttons, drop down menus, fields for receiving information from a user, and the like. In one aspect, such actions can include editing or changing the selected region of the graphical representation or other search criteria. In certain aspects, the system further includes a result formatter for formatting search results (e.g., to build appropriate user interfaces such as Web pages, to specify links, provide a way to associate actions (e.g., “delete,” “edit,” etc.) with images, text, hyperlinks and/or other displays).

In certain aspects, results of a search query may be linked to option fields allowing a user to order items associated with an object. For example, a checkbox may be included next to a probe or group of probes to allow a user to add them to a shopping cart or directly order the probe group. Similarly, selecting an array design may cause the system to display options to purchase the array design. In certain aspects, the system may display items associated with objects that have relationships to objects associated with items being purchased. For example, if a user selects a Probe Group 1 for purchase, the system would display one or more array layouts that have included Probe Group 1 and/or reagents (e.g., such as controls, probes, labeling reagents, amplification reagents) that other users who have selected Probe Group 1 have purchased or which otherwise may be of interest to the user.

A variety of interfaces may be used to implement the functions of the system. In one embodiment, in the case of web applications, a servlet container uses an HTTP Session interface to create a session between an HTTP client and an HTTP server. The session may persist for a specified time period, across more than one connection or page request from a user. In one aspect, one user may be involved in a session, and the user may visit the web application many times. However, multiple users also may be involved in a session. The server can maintain a session in many ways, such as by using cookies or rewriting URLs.

In certain embodiments, the system comprises a session manager. The session manager acts as a factory class that may be used to generate objects, and in one aspect, related objects when a user interacts with the system. In another embodiment, information relating to all user sessions is maintained in a collection within the session manager. In a further embodiment, one session manager instance is associated with one application in the system. In still a further embodiment, session instances are associated with session manager instances. This structure ensures that there are collections of instances per application in the system.

The session manager may have one or more of the following properties. The session manager may comprise a collection of all Session objects for all current users using the system or an application of the system. In one aspect, the collection is in the form of a Hashtable.

In one embodiment, the system contains a plurality of different application objects. Application objects comprise object representations of underlying database tables. In one aspect, each application has a context associated with it. Context is a logical area of the application, which contains the configuration information for the application. This information can be accessed within that application via this context.

For example, in one embodiment, the system comprises an application bootstrap framework, which comprises a set of classes and a configuration file. In one aspect, the configuration file contains configuration information for each application. The application bootstrapping mechanism starts working when the system starts up for the first time. When system starts up, a system initialization program (e.g., start up Servlet) instantiates an instance of Application object per application in the system. The first request to the application server will check whether application context for the named application is there or not. If application context is not present then it creates one. In one aspect, the application bootstrap framework communicates with an object relationship mapping means in the system, assisting a user to identify object categories associated with a user query. In another aspect, in response to the identification of object categories, an output (e.g., such as a display on a graphical user interface) is generated

In one embodiment, the system includes an event generation and processing framework. Whenever an action takes place on an object in the system, the system generates an event. The object that generates this event is called as the event source. In one aspect, when events occur, a user with requisite permissions is notified for these events. In certain aspects, to get an event notification, the user must register him/herself for that type of event. The user will get notifications:only for those types of events for which the user has registered. For this, the system maintains a queue of the events, which contains only those events for which at least one user has registered. This queue is then processed periodically and notifications are sent to the users, e.g., by email. In one embodiment, the event notification framework generates events and adds them to the event queue, while the event processing framework processes the events from the event queue and then sends the notifications. See FIG. 7.

In one aspect, events supported by system application(s) are pre-configured. For example, the system memory can include a database of all supported (e.g., pre-configured events). In one aspect, the database includes a table comprising an event ID uniquely identifying a supported event (e.g., an annotation update), an action name for the event (e.g., “Annotation Update”), and name of an action that will be executed during post-processing of an event. The table may be a hashtable collection which may be associated with a particular user session by a session ID. In one aspect, the event manager allows a user to create, add and/or notify a user about events.

The Event Manager may include a mechanism for providing an output to a user which may include, but is not limited to the name of the event, an ID for an event uniquely identifying the event in the database, date of the event, content of an message to the user describing the event, type of event (e.g., triggered or periodic), and the like.

In certain aspects, a user may have an event manager associated with that particular user's events.

In a further aspect, the system comprises a Hashtable collection which contains a key-value pair of application name and session manager instance associated with an application. This collection is useful for identifying session manager instances for all applications in the system.

In one embodiment, a system according to the invention creates a session manager for an application if one did not already exist. In one aspect, the system may output data relating to all the session manager instances that are associated with the system (e.g., for all applications of the system). Similarly, the system may output information relating to the collection of session instances associated with any given session manager. The system may further remove a session from a session collection as well as invalidate a user session.

The system of the invention may include a functional element that produces an array layout which includes one or more probes obtained by the probe developer (also called an array layout developer). The array layout developer may be configured to develop a chemical array layout in response to information received from one or more users, where the information received from the one or more users typically includes array request information. By “array layout” is meant a collection of information, e.g., in the form of a file, that represents the location of probes that have been assigned to specific features of an array format. The phrase “array format” refers to a format that defines an array by feature number, feature size, Cartesian coordinates of each feature, and distance that exists between features within a given array. The phrase “array request information” is use broadly to encompass any type of information/data that is employed in developing an array layout, where representative types of array request information include, but are not limited to: probe content identifiers (e.g., in the form of probe sequence, e.g., as determined by the probe developer functional element of the subject systems), gene/protein name, accession number, annotation, etc.; array function information (e.g., in the form of types of genes/proteins to be studied using the array); array format information (e.g., feature number, feature size, Cartesian coordinates of each feature, and distance that exists between features within a given array); etc. As such, the array layout developer of the processing modules of the subject systems is a functional element that produces an array layout in response to receiving array request information.

In certain embodiment, the system further includes an instructional module that executes instructions from a computer program product for displaying Web pages that instruct a user how to use and interact with the system. In one aspect, the instructional module provides a tutorial page, explaining the purpose of the module (e.g., to provide instructions for obtaining probes for a region of interest and/optionally designing and/or ordering arrays). Additional Web pages or sections of web pages can be provided to describe and provide examples of various system functions (e.g., such as searching, uploading biopolymeric sequences and/or probes, downloading probes, etc.) and can provide interactive sessions to illustrate system functions. Such sessions can include displaying information relating to searching for information about probes, identifying probes, uploading probes, downloading probes, demonstrating sorting, viewing, saving search results, providing tutorials for generating an array layout, and the like. The instructional module can include a variety of graphics, including text, images, animation and can also provide accompanying voiceovers.

### Representative Methods

The methods of the invention are drawn to obtaining at least one probe that corresponds to a region of a biopolymeric sequence that is selected using graphical visualization and selection functions. In general, embodiments of the subject methods include the following steps: (a) submitting a biopolymer request to a system of the invention comprising a graphical representation of a biopolymeric sequence; and (b) obtaining at least one probe from the system in response to the request.

In certain embodiments, a graphical representation is generated by the system and displayed to the user and the user selects a region of the graphical representation for which probes are sought.

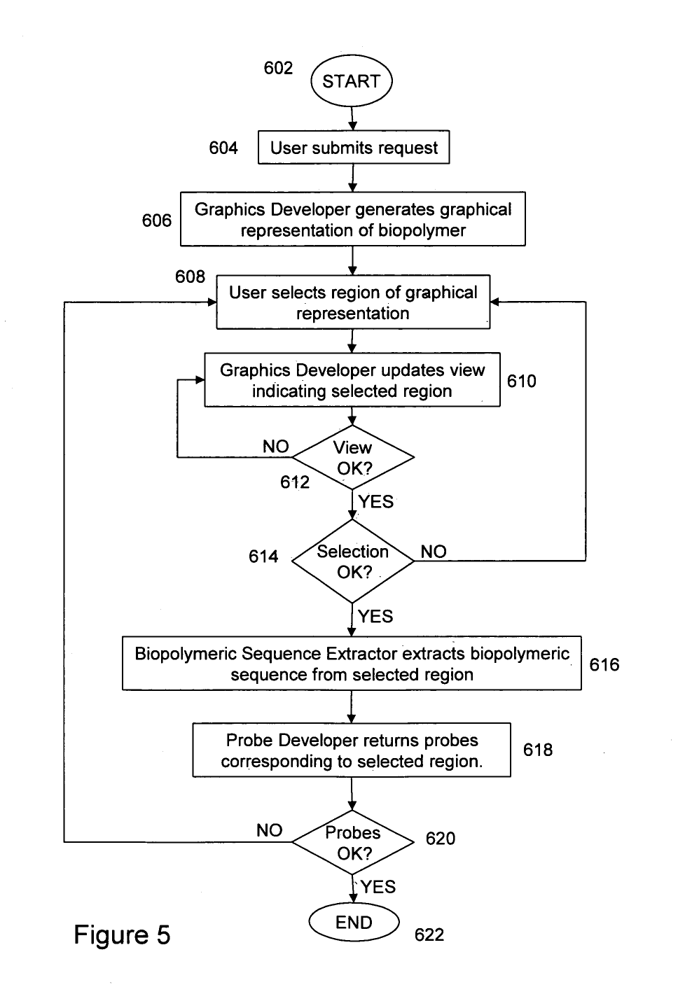

Below is a discussion of several embodiments of the methods of the subject invention. FIG. 5 provides a block diagram of a particular embodiment of the methods of the invention, the elements of which will be referred to in the discussion below. It is to be understood that methods of the invention may have fewer or additional steps as shown in FIG. 5.

In certain embodiments, a user submits a biopolymer request to the system (604) using GUI (512) of computer (510). In general, the biopolymeric request identifies a biopolymer of interest for which probes are sought for at least a region thereof. The biopolymer identifier can be any of a variety of known biopolymer identifiers, such as an organism and an chromosome number, a gene accession number, or even the biopolymeric sequence itself. The biopolymer of interest can be any biopolymer, where in certain embodiments the biopolymer is a nucleic acid sequence while in other embodiments the biopolymeric sequence is an amino acid sequence. Submitting a biopolymer request may be done using any convenient method, including inputting a biopolymeric sequence directly into a sequence input window displayed to the user on the GUI or by instructing the system to retrieve the sequence from a public or private database. For example, if a user wants to obtain probes for a specific gene of interest, he may input the GenBank accession number and instruct the system to retrieve the sequence. The system may be configured to accept any number of biopolymeric sequence identifiers for use in retrieving a sequence of interest, including organism (e.g., human, mouse, E. coli, C. elegans, D. melanogaster, etc.), chromosome number, chromosomal location, gene or protein identifier (e.g., GenBank or SwissProt accession number), or functional property.

In certain embodiments, the system generates a graphical representation of the submitted biopolymer (606) (e.g., using the graphics developer (532)). In certain embodiments, the graphics developer is pre-programmed to generate a graphical representation without input from the user whereas in other embodiments, the user is prompted to provide guidance as to features of the graphical representation to be returned (e.g., magnification, number and/or types of views, annotations, etc.).

The graphical representation can take many forms. In certain embodiments, the graphical view represents the primary sequence of the biopolymer. In certain of these embodiments, the graphical representation has annotations associated with it, including positional markers (e.g., nucleotide or amino-acid number), structural identifiers (e.g., introns, exons, active sites, functional domains, etc.), organism, type of biopolymer, and/or other relevant information. In other embodiments, the graphical view represents certain secondary structural characteristics. For example, if the biopolymeric sequence is a nucleic acid, the graphical representation can indicate regions of secondary structure, such as hairpin loops or ribiozyme-like structures. In certain embodiments, the graphical view represents tertiary structural characteristics. For example, in embodiments in which the biopolymeric sequence is a protein, a three dimensional representation may be generated that shows regions with beta-sheet, beta-barrel, helical, or other structural features. In certain embodiments, the user can alternate between these views or view more than one at a time.

As indicated in the previous section, in certain embodiments, the graphical representation is manipulable by the user. For example, the user can zoom in and out, flip, rotate, change the color, add or remove annotation, etc. These manipulations may occur in real time or by inputting the desired graphical view changes into the system and submitting a request the graphics developer to implement the changes.

Once a suitable graphical representation of the biopolymeric sequence has been displayed, which in certain embodiments is decided by the user, the user selects at least one region on the graphical representation for which probes are to be obtained by the system (608). The number of regions selected by the user can vary widely, and as such there is no limitation in this regard other than the size of the submitted biopolymer. The user employs GUI (512) of computer (510) to accomplish this, in certain embodiments using “drag and click” functions most often implemented by manipulating a computer mouse (although there are many computer mouse-independent ways in which a user can select a region of the graphical representation).

In certain embodiments, the graphics developer is configured to update the graphical representation to indicate the user selected region (610) either automatically or at the request of the user. The selected region can be indicated on or by the graphical representation in any number of ways including, but not limited to, altering the color of the selected region to be distinct from the color of the un-selected region, marking the beginning and ending of the selected region with hash marks (or other graphical markers), generating an independent graphical view of the selected region that is displayed concurrently with the graphical representation of the entire biopolymeric sequence, etc.

In certain embodiments, a graphical representation is shown which takes advantage of zooming capabilities to provide differing level views of the biopolymer of interest.

For example, high level, medium level and detailed views may be provided to a user on the same GUI in separate viewer windows (e.g., high-, mid-, and low-level view windows). The high level view can represent the entire biopolymer of interest, but is not limited to such. By selecting a region of the high level view of the biopolymer with a cursor mechanism, a mid-level view of the selected region of the biopolymer appears in a second viewer window to show it in greater detail.

An indicator such as a box, line, highlight, etc., indicates the selection of the high level view to be magnified in the mid-level view. It is also possible to select more than one region of the high level view for viewing in the mid-level view.

In certain embodiments, the a user employs the mid level view to more carefully select a region of interest for which probes are sought, as the high level view may not have sufficient resolution. In these embodiments, the user selects a region on the mid level view (similar to selecting a region or regions on the high level view), which is displayed in the low level view window. The low level (or detailed) view may show specific details as to genes and annotations of the selected region in the high and/or mid level views (e.g., regulatory regions, structural motifs, etc.). In certain embodiments, some or all of these annotations may also be shown on the high and/or mid level views.

In certain embodiments, all three viewers are interlinked so that each is updated whenever a manipulation of any of the viewers is performed. In these embodiments, because all viewers are interactively linked, a user can click on any portion of any single viewer of the display and the corresponding features in the other viewers will be automatically and simultaneously updated. In this way a selection of an element in the low level view can be immediately seen in the context of both the mid and high level view.

In certain embodiments,.additional supplementary information about a biopolymeric sequence may be made available via tooltips or popup dialogs. For example, by hovering over a region of a biopolymeric sequence (or a selected region thereof, a tooltip may be displayed with additional details about the biopolymeric sequence. For example, such dialog may include information about the chromosomal location of the biopolymeric sequence and/or certain supporting information that might be relevant (e.g., expression data). The supplementary details may come from local databases or external databases (e.g., web sites, etc.).

The process of selecting a region on the graphical representation may take several iterations of viewing, selecting, and updating (612 and 614) until the user accepts the graphical representation view and the selection thereof.

Once the region (or regions) for which a probe is to be obtained is selected, the user submits the selection to the system. In certain embodiments, submitting the selected region involves selecting (or clicking on) a “submit” icon on the window displayed to the user on the GUI (512) (e.g., using a computer mouse). In certain other embodiments, the user strikes a key on a keyboard in operative communication with the computer (510) to indicate that a region of interest has been selected. In certain embodiments, the biopolymeric sequence that corresponds to the graphically selected region is extracted using the biopolymeric sequence extractor of the system (616). In these embodiments, the system correlates the graphical selection on the graphical representation with the biopolymeric sequence submitted by the user (in step 604). In certain other embodiments, the system is configured to correlate the user-selected region(s) of the graphical representation with the biopolymeric sequence in real time during the selection step, and as such an independent biopolymeric sequence extraction step is not necessary.

The biopolymeric sequence that corresponds to the user-selected region(s) of the graphical representation of the biopolymeric sequence is submitted to the probe developer which is configured to obtain at least one probe that corresponds (or is specific for) the selected region(s) (618). Representative configurations of the probe developer are discussed in detail in the previous section. In certain embodiments, the probe(s) returned by the probe developer are nucleic acid probes (e.g., oligonucleotides, polynucleotides, cDNA, etc.) whereas in other embodiments the probes returned are amino acid probes (e.g., poly-peptides, proteins, antibodies, etc.). In certain embodiments the user will indicate which type of probe is to be returned form the system.

The probes obtained by the probe developer are displayed to the user. The probes may be displayed in any variety of ways, including as a simple list of probes (e.g., a table with probe information therein, in certain embodiments including the biopolymeric sequence of the probe), graphical representations of the biopolymerice probes (e.g., the probes can be graphically represented, in certain embodiments indicating where the probes bind along the graphical representation of the selected region of interest), or both. In certain embodiments, the view of the obtained probe(s) provides the user with executable functions, including selecting/deselecting probes, toggling between graphical and table views of the probes, etc. In certain embodiments, the user views the returned results and decides which probes, if any, to select for further processing or use (620).

In certain embodiments, the user can reject the entirety of the returned probe(s) and select a different region for which to obtain a probe(s). For example, the system may return too many probe sequences and the user may decide to reduce the size of the selected region so that fewer probes are obtained. Conversely, the system may obtain too few probes, whereby the user may select a larger region of the biopolymeric sequence. As such, the system provides a mechanism for reiterating selection and probe obtainment in a rapid manner, thereby allowing a use greater flexibility and speed in refining the selection of regions of a biopolymeric sequence for which probes are sought.

In certain embodiments, upon viewing the search results, a user can select individual or groups of probes from the obtained set and add them to a collection in a database. A user can select or remove probes from this database collection. In this way a user can generate database collections of probes derived from numerous selected regions of a biopolymeric sequence or from selected regions of distinct biopolymeric sequences. In certain embodiments, the system will create databases of probes independent of the user, in certain embodiments allowing a user to view the collections and the search parameters at a later time.

In certain embodiments, the methods include saving a version of the probe sequence group that is output by the system. In certain embodiments, the method includes modifying a version of the probe sequence group. The resultant modified version may be saved as a new version. In certain embodiments, the method includes saving multiple versions of the probe sequence group, where the multiple versions may or may not be compared. In certain embodiments, one or more permitted users of the system are allowed to view and modify different versions of a probe sequence group. In certain embodiments, the methods include selecting a version of the probe sequence group. The version may be selected, e.g., for ordering, as discussed below.

In certain embodiments, the methods include ordering synthesized probe(s) that include the sequences of the selected probe group. In certain embodiments, the synthesized probes are synthesized on an array. In certain embodiments, the inputting is via a graphical user interface in communication with the system.

In certain embodiments, the user may choose to obtain an array having the generated probe present therein. As such, the generated probe can be included in an array layout, and an array fabricated according to the array layout that includes the generated probe. In certain embodiments, the user may specify the location of the probe in the product layout. Specifying may include choosing a particular location in a given layout, or choosing from a section of system-provided array layout options in which the probe is present at various locations. Array fabrication according to an array layout can be accomplished in a number of different ways. With respect nucleic acid arrays in which the immobilized nucleic acids are covalently attached to the substrate surface, such arrays may be synthesized via in situ synthesis in which the nucleic acid ligand is grown on the surface of the substrate in a step-wise fashion and via deposition of the full ligand, e.g., in which a presynthesized nucleic acid/polypeptide, cDNA fragment, etc., onto the surface of the array.

As summarized above, the systems of the invention receive probe request information from a user and generate one or more probe sequence groups therefrom, where the output of the system may be individual probes, collections of probes, or even array layouts that include the generated probes. The generated probe sequence groups are, in representative embodiments, forwarded to the user for evaluation and use. Accordingly, in certain embodiments, the system determines a group of probe sequences, i.e., a probe group, e.g., for inclusion on a chemical array. The probe group may belong to a common annotation category (e.g., a selected region of a biopolymer). In certain embodiments, the one or more probe groups is modifiable by one or more permitted users of the system. In certain embodiments, versions of probe groups may be stored in the memory of the system (e.g., as a database). In certain embodiments, the system includes a difference engine for comparing one or more versions of the probe groups. In certain embodiments, the output manager displays results of any comparing step.

As such, the systems find use in at least generating probes for use on arrays, and in certain embodiments are employed in the generation of array layouts. In such embodiments, the array layouts generated by the subject systems can be layouts for any type of chemical array, where in representative embodiments the array layouts are layouts for biopolymeric arrays, such as nucleic acid and amino acid arrays. In representative embodiments, the layouts generated by the subject systems are for nucleic acid arrays.

In certain embodiments, the systems include an array layout functionality, as described in copending application Ser. No. 11/001,700 (attorney docket number 10041581-1) titled “Systems and Methods for Producing Chemical Array Layouts,” and filed on even date herewith. In certain of these embodiments, the system includes an array layout developer, where the array layout developer includes a memory having a plurality of rules relating to array layout design and is configured to develop an array layout based on the application of one or more of the rules to information that includes array request information received from a user.

In certain embodiments, the output manager further provides a user with information regarding how to purchase the identified at least one probe sequence. In certain embodiments, the information is provided in the form of an email. In certain embodiments, the information is provided in the form of web page content on a graphical user interface in communication with the output manager. In certain embodiments, the web page content provides a user with an option to select for purchase one or more synthesized probe sequences. In certain embodiments, the web page content includes fields for inputting customer information. In certain embodiments, the system can store the customer information in the memory. In certain embodiments, the customer information includes one or more purchase order numbers. In certain embodiments, the customer information includes one or more purchase order numbers and the system prompts a user to select a purchase order number prior to purchasing the one or more synthesized probe sequences.

In certain embodiments, in response to the purchasing, the one or more probe sequences are synthesized on an array. With respect to actual array fabrication, in certain embodiments, the user may himself produce an array having the generated array layout. In yet other embodiments, the user may forward the array layout to a specialized array fabricator or vendor, which vendor will then fabricate the array according to the array layout.

In yet other embodiments, the system may be in communication with an array fabrication station, e.g., where the system operator is also an array vendor, such that the user may order an array directly through the system. In response to receiving an order from the user, the system will forward the array layout to a fabrication station, and the fabrication station will fabricate the array according to the forwarded array layout.

Arrays can be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, light directed fabrication methods may be used, as are known in the art. Interfeature areas need not be present particularly when the arrays are made by light directed synthesis protocols.

Following array fabrication, the fabricated array may then be forwarded, i.e., shipped, to the user using any convenient means. As such, following fabrication, one or more array units may then be forwarded to one or more remote customer stations.

Chemical arrays having probes generated by the subject systems and methods find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992. Also of interest are U.S. Pat. Nos. 6,656,740; 6,613,893; 6,599,693; 6,589,739; 6,587,579; 6,420,180; 6,387,636; 6,309,875; 6,232,072; 6,221,653; and 6,180,351. In certain embodiments, the subject methods include a step of transmitting data from at least one of the detecting and deriving steps, as described above, to a remote location.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934. However, arrays may be read by any other method or apparatus than the foregoing, with other reading methods including other optical techniques (for example, detecting cheiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

The invention also provides programming, e.g., in the form of computer program products, for use in practicing the methods. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithrms for carrying out the above described methodology.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A system for obtaining at least one probe corresponding to a selected region of a biopolymeric sequence, said system comprising:
(a) an input manager for receiving a user biopolymer request, wherein said user biopolymer request comprises a graphical representation of a biopolymeric sequence;
(b) a probe developer for obtaining at least one probe corresponding to a selected region of said graphical representation of said biopolymeric sequence;
(c) an output manager for providing said at least one obtained probe.

2. The system according to claim 1, wherein said system further comprises a graphical user interface (GUI) for transferring information between said system and a user.

3. The system according to claim 1, wherein said system further comprises a graphics developer for generating at least one graphical representation of a biopolymeric sequence in-response to identifier of said sequence.

4. The system according to claim 3, wherein said graphics developer is configured to generate graphical representations that can be manipulated by a user.

5. The system according to claim 3, wherein said graphics developer is configured to automatically update said graphical representation of said biopolymeric sequence in response to a user selecting a region of said graphical representation of said biopolymeric sequence.

6. The system according to claim 1, wherein said probe developer is configured to obtain said probe by using a probe design algorithm.

7. The system according to claim 1, wherein said probe developer is configured to obtain said probe by retrieving a probe from a database.

8. The system according to claim 1, wherein said output manager further provides probe content and associated annotation information relating to said at least one probe corresponding to said selected region.

9. The system according to claim 1, wherein said system provides for remote communication between a user and said system.

10. The system according to claim 1, wherein said system provides for communication between a user and said system via the Internet.

11. The system according to claim 1, wherein said system obtains a group of probe sequences corresponding to said selected region.

12. The system according to claim 11, wherein said system obtains a plurality of probe groups corresponding to said selected region.

13. The system according to claim 1, wherein said output manager provides said user with an option to select for purchase one or more of said obtained probes.

14. The system according to claim 13, wherein in response to said purchasing the one or more probe sequences are synthesized on an array.

15. A method for obtaining at least one probe corresponding to a selected region of a biopolymeric sequence, said method comprising:
(a) submitting a biopolymer request to a system according to claim 1, wherein said biopolymer request comprises a graphical representation of a biopolymeric sequence; and
(b) obtaining at least one probe from said system in response to said request.

16. The method according to claim 15, wherein said method further comprises viewing at least one graphical representation of said biopolymeric sequence and selecting at least one region of said graphical representation.

17. The method according to claim 15, wherein said biopolymeric sequence is a nucleic acid sequence.

18. The method according to claim 17, wherein said nucleic acid is at least a part of a chromosome.

19. The method according to claim 15, wherein said biopolymeric sequence is an amino acid sequence.

20. The method according to claim 15, wherein said probe is a nucleic acid.

21. The method according to claim 15, wherein said probe is a polypeptide.

22. The method according to claim 15, wherein said region is selected by highlighting said region on said graphical representation of said nucleic acid.

23. The method according to claim 15, wherein said graphical representation is manipulatable.

24. The method according to claim 22, wherein said highlighted region is represented as a distinct graphical representation from said graphical representation of said nucleic acid.

25. The method according to claim 24, wherein said distinct graphical representation is a distinct color.

26. The method according to claim 15, wherein said obtaining step comprises submitting said selected region to a nucleic acid probe database.

27. The method according to claim 15, wherein said obtaining step comprises submitting said selected region to a nucleic acid probe design algorithm.

28. The method according to claim 15, wherein said submitting is via the Internet.

29. The method according to claim 13, wherein any of said steps are reiterated.

30. The method according to claim 15, further comprising selecting at least one of said obtained probes and saving it in a database.

31. The method according to claim 30, further comprising adding probes to said database.

32. The method according to claim 30, further comprising deleting probes from said database.

33. The method according to claim 30, wherein probes obtained from different selected regions are added to said database.

34. The method according to claim 15, wherein multiple regions of said biopolymeric sequence are selected on said graphical representation.

35. The method according to claim 16, wherein the binding location of said at least one probe is indicated graphically on said graphical representation of said selected region.

36. A method for obtaining at least one probe corresponding to a selected region of a biopolymeric sequence, said method comprising:
(a) receiving a biopolymer request that comprises a graphical representation of a biopolymeric sequence; and
(b) obtaining at least one probe in response to said request.

37. A computer program product comprising a computer readable storage medium having a computer program stored thereon, wherein said computer program, when loaded onto a computer, operates said computer to:
a) receive a biopolymer request that comprises a graphical representation of a biopolymeric sequence; and
b) obtain at least one probe in response to said request.
